(19) United States
(12) Patent Application Publication
(10) Pub. No.: US 2011/0118114 A1
(43) Pub. Date: May 19, 2011

(54) COLOR STABLE SUPERABSORBENT

(75) Inventors: Ulrich Riegel, Landstuhl (DE); Thomas Daniel, Waldsee (DE); Norbert Herfert, Altenstadt (DE); Mark Elliott, Ludwigshafen (DE)

(73) Assignee: BASF SE, Ludwigshafen (DE)

(21) Appl. No.: 13/054,287

(22) PCT Filed: Jul. 29, 2009
(86) PCT No.: PCT/EP09/59793

§ 371 Date: Jan. 14, 2011

(30) Foreign Application Priority Data
Jul. 31, 2008 (EP) 08161572.6

(51) Int. Cl. B01J 20/22; C07C 313/02

(52) U.S. Cl. 502/401; 562/125; 562/126

(57) ABSTRACT

Superabsorbents comprising a compound of the formula (I)

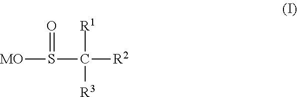

in which
M is a hydrogen atom, an ammonium ion, a monovalent metal ion or one equivalent of a divalent metal ion of groups 1, 2, 8, 9, 10, 12 or 14 of the periodic table of the elements;
R¹ is OH or NR⁴R⁵ where R⁴ and R⁵ are each independently H or C₁-C₆-alkyl;
R² is H or an alkyl, alkenyl, cycloalkyl or aryl group, where this group optionally has 1, 2 or 3 substituents which are each independently selected from C₁-C₆-alkyl, OH, O—C₁-C₆-alkyl, halogen and CF₃; and R³ is COOM, SO₃M, COR⁴, CONR⁴R⁵ or COOR⁴, where M, R⁴ and R⁵ are each as defined above or, when R² is aryl which is optionally substituted as specified above, are also H,
   salts thereof or mixtures of such compounds and/or salts thereof,
exhibit improved stability to discoloration in the course of storage under elevated temperatures and/or elevated air humidity.

The present invention relates to a color-stable superabsorbent, to a process for producing it and to the use thereof and to hygiene articles comprising it. A color-stable superabsorbent is understood to mean a superabsorbent which is discolored only to a minor degree, if at all, in the course of storage under elevated temperature and air humidity.

Superabsorbents are known. For such materials, names such as “high-swellability polymer”, “hydrogel” (often also used for the dry form), “hydrogel forming polymer”, “water-absorbing polymer”, “absorbent gel-forming material”, “swellable resin”, “water-absorbing resin” or the like are also in common use. The substances in question are crosslinked hydrophilic polymers, especially polymers of (co)polymerized hydrophilic monomers, graft (co)polymers of one or more hydrophilic monomers on a suitable graft base, crosslinked cellulose ethers or starch ethers, crosslinked carboxymethylcellulose, partly crosslinked polyalkylene oxide or natural products which are swellable in aqueous liquids, for example guar derivatives, of which water-absorbing polymers based on partly neutralized acrylic acid are the most widespread. The essential properties of superabsorbents are their abilities to absorb several times their own weight of aqueous liquids and not to release the liquid again even under a certain pressure. The superabsorbent, which is used in the form of a dry powder, is converted to a gel when it absorbs liquid, and correspondingly to a hydrogel when it absorbs water as usual. Crosslinking is essential for synthetic superabsorbents and is an important difference from customary pure thickeners, since it leads to the insolubility of the polymers in water. Soluble substances would not be usable as superabsorbents. By far the most important field of use of superabsorbents is the absorption of body fluids. Superabsorbents are used, for example, in diapers for infants, incontinence products for adults or feminine hygiene products. Other fields of use are, for example, as water-retaining agents in market gardening, as water stores for protection from fire, for liquid absorption in food packaging, or quite generally for absorbing moisture.

Superabsorbents can absorb several times their own weight of water and retain it under a certain pressure. In general, such a superabsorbent has a CRC (“centrifuge retention capacity”, see below for test method) of at least 5 g/g, preferably at least 10 g/g and more preferably at least 15 g/g. A “superabsorbent” may also be a mixture of different individual superabsorbent substances or a mixture of components which exhibit superabsorbent properties only when they interact; it is not so much the substance composition as the superabsorbent properties that are important here.

What is important for a superabsorbent is not just its absorption capacity but also the ability to retain liquid under pressure (retention) and liquid transport in the swollen state. Swollen gel can hinder or prevent liquid transport to as yet unswollen superabsorbent (“gel blocking”). Good transport properties for liquids are possessed, for example, by hydrogels which have a high gel strength in the swollen state. Gels with only a low gel strength are deformable under an applied pressure (body pressure), block pores in the superabsorbent/cellulose fiber suction body and thus prevent further absorption of liquid. An increased gel strength is generally achieved through a higher degree of crosslinking, which, however, reduces the absorption capacity of the product. An elegant method of increasing the gel strength is that of increasing the degree of crosslinking at the surface of the superabsorbent particles compared to the interior of the particles. To this end, superabsorbent particulars which have usually been dried in a surface postcrosslinking step and have an average crosslinking density are subjected to additional crosslinking in a thin surface layer of the particles thereof. The surface postcrosslinking increases the crosslinking density in the shell of the superabsorbent particles, which raises the absorption under compressive stress to a higher level. While the absorption capacity in the surface layer of the superabsorbent particles falls, their core, as a result of the presence of mobile polymer chains, has an improved absorption capacity compared to the shell, such that the shell structure ensures improved liquid conduction, without occurrence of gel blocking. It is likewise known that superabsorbents which are relatively highly crosslinked overall can be obtained and the degree of crosslinking in the interior of the particles can subsequently be reduced compared to an outer shell of the particles.

Processes for producing superabsorbents are also known. Superabsorbents based on acrylic acid, which are the most common on the market, are produced by free-radical polymerization of acrylic acid in the presence of a crosslinker (the “interior crosslinker”), the acrylic acid being neutralized to a certain degree before, after or partly before and partly after the polymerization, typically by adding alkali, usually an aqueous sodium hydroxide solution. The polymer gel thus obtained is comminuted (according to the polymerization reactor used, this can be done simultaneously with the polymerization) and dried. The dry powder thus obtained (the “base polymer”) is typically postcrosslinked on the surface of the particles, by reacting it with further crosslinkers, for instance organic crosslinkers or polyvalent cations, for example aluminum (usually used in the form of aluminum sulfate) or both, in order to obtain a more highly crosslinked surface layer compared to the particle interior.

A problem which often occurs in the case of superabsorbents is discoloration, which occurs in the course of storage under elevated temperature or elevated air humidity. Such conditions often occur in the case of storage of superabsorbents in tropical or subtropical countries. Superabsorbents tend to yellow under such conditions; they may even assume a brown or even almost black color. This discoloration of the actually colorless superabsorbent powder is unsightly and undesired, since it is visible especially in the desired thin hygiene products, and consumers reject unsightly hygiene products. The cause of the discoloration has not been entirely clarified, but reactive compounds such as residual monomers from the polymerization, the use of some initiators, impurities in the monomer or the neutralizing agent, surface postcrosslinkers or stabilizers in the monomers used appear to play a role.

Fredric L. Buchholz and Andrew T. Graham (eds.) give, in: “Modern Superabsorbent Polymer Technology”, J. Wiley & Sons, New York, U.S.A. /Wiley-VCH, Weinheim, Germany, 1997, ISBN 0-471-19411-5, a comprehensive overview of superabsorbents, properties thereof and processes for producing superabsorbents.

WO 2008/055856 A1 teaches the prevention of discoloration of a superabsorbent which is caused by an excessively high iron content of sodium hydroxide solution which is used for partial neutralization of the acrylic acid in the course of preparation of the superabsorbent, by adding phosphoric acid or phosphate salts. JP 05/086 251 A teaches the use of phosphoric acid derivatives or salts thereof, especially 1-hydroxyethylidene-1,1-diphosphonic acid, ethylenediaminetetra(methylenephosphonic acid), diethylenetriaminepenta(methylenephosphonic acid) or the alkali metal or ammonium salts thereof as stabilizers of superabsorbents against discoloration. WO 03/059 962 A1 or the equivalent patent application US 2005/0085604 A1 discloses the use of metal chelating agents in any step of superabsorbent production, and the addition of a reducing or oxidizing agent before drying of the water-containing polymer as measures against discoloration. WO 03/014 172 A2 relates to the use of superabsorbents formed from high-purity acrylic acid, which have been freed especially of aldehydes to prevent discoloration of the superabsorbents. WO 00/55245 A1 teaches the stabilization of superabsorbents against discoloration by treatment with an inorganic reducing agent and optionally a metal salt. The inorganic reducing agent is typically a hypophosphite, phosphite, bisulfite or sulfite. The metal salt is typically a colorless (the property of “colorless” is often also simply referred to as “white”) phosphate, acetate or lactate, but not a halide. According to the teaching of WO 2006/058 682, discoloration of superabsorbents is avoided when the drying and the postcrosslinking reaction are carried out in an atmosphere which is essentially free of oxidizing gases.

EP 505 163 A1 discloses the use of a combination of surface-active substance and a compound which adds onto double bonds, for example unsubstituted or substituted alkyl- or arylsulfinic acids or salts thereof to reduce the level of residual monomers in superabsorbents. EP 668 080 A2 and the partial application EP 1570 869 A1 relate to the use of organic acids, including sulfinic acids, but exclusively of salts of organic acids or sulfinic acids, or of polyamino acids or salts thereof, for reducing the level of residual surface postcrosslinker, especially of epoxy compounds used as surface postcrosslinkers, after the surface postcrosslinking. According to the teaching of EP 386 897 A2, EP 441 975 A1 and EP 605 215 A1 teach the use of sulfites, hydrogensulfites or thiosulfates to reduce the level of residual monomers from the polymerization. EP 1 645 596 A1 teaches the stabilization of superabsorbents against discoloration by addition of an inorganic salt, of an aminocarboxy acid chelating agent and of an organic antioxidant. The inorganic salts used are sulfites, bisulfites, pyrosulfites, dithionites, trithionates, tetrathionates, thiosulfates or nitrites. EP 1 577 349 A1 teaches the use of these salts for the same purpose, although the iron content of the superabsorbents treated therewith is kept below 1 ppm by weight. US 2009/0023848 A1 discloses the treatment of a superabsorbent with an antioxidant, for example 2-hydroxysulfinatoacetic acid, for the purpose of stabilization against discoloration.

EP 1 199 315 A2 teaches the use of a redox initiator system for initiating a polymerization reaction, said redox initiator system comprising, as the reducing component, a sulfinic acid or a sulfinate, especially 2-hydroxysulfinatoacetic acid or a salt thereof. WO 99/18067 A1 discloses particular hydroxyl- or aminoalkyl- or arylsulfinic acid derivatives or mixtures thereof and the use thereof as reducing agents which do not eliminate formaldehyde. WO 2004/084 962 A1 relates to the use of these sulfinic acid derivatives as the reducing component of a redox initiator for polymerization of partly neutralized acrylic acid to superabsorbents. Initiators are used in a small amount, since excessively large amounts of initiator lead to undesirably short polymer chains and, as a result, an undesirably high content of extractables in the polymer. The constituents of a redox initiator are additionally depleted in the course of polymerization, show no effect in the finished polymer and are normally undetectable in the finished polymer. In the case of some initiators, however, it is also possible for colored reaction products which remain in the polymer to form in an undesired manner. In general, a few routine tests are sufficient to find suitable initiators.

The prior international patent application PCT/EP2008/051009 teaches the addition of a basic salt of a divalent metal cation to superabsorbents, in order to increase the stability to discoloration among other reasons. The prior international patent application PCT/EP2008/051010 discloses the use of carboxylic salts and/or basic salts of trivalent metal cations for the same purpose.

It is an object of the present invention to find other superabsorbents or superabsorbents which are stabilized even better to discoloration, especially to yellowing or browning in the course of storage under elevated temperature and/or elevated air humidity. If at all, this should only insignificantly impair the use properties of the superabsorbent, especially its absorption capacity for fluid, including under pressure, and its ability to conduct fluid, but also its free flow. Further objects of the invention are the finding of a process for producing such a superabsorbent, and uses of this superabsorbent.

This object is achieved by a superabsorbent comprising a compound of the formula (I)

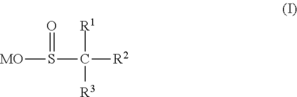

in which

M is a hydrogen atom, an ammonium ion, a monovalent metal ion or one equivalent of a divalent metal ion of grows 1, 2, 8, 9, 10, 12 or 14 of the periodic table of the elements;

R¹ is OH or NR⁴R⁵ where R⁴ and R⁵ are each independently H or C₁-C₆-alkyl;

R² is H or an alkyl, alkenyl, cycloalkyl or aryl group, where this group optionally has 1, 2 or 3 substituents which are each independently selected from C₁-C₆-alkyl, OH, O—C₁-C₆-alkyl, halogen and CF₃; and

R³ is COOM, SO₃M, COR⁴, CONR⁴R⁵ or COOR⁴, where M, R⁴ and R⁵ are each as defined above or, when R² is aryl which is optionally substituted as specified above, are also H,

salts thereof or mixtures of such compounds and/or salts thereof.

Additionally found have been a process for producing this superabsorbent, uses of this superabsorbent and hygiene articles which comprise this superabsorbent.

The inventive superabsorbents which comprise sulfinic acid derivatives described by the formula exhibit surprisingly good stability to discoloration, without their use properties being impaired.

In the above formula, alkyl represents straight-chain or branched alkyl groups which have preferably 1-6 and especially 1-4 carbon atom. Examples of alkyl groups are methyl, ethyl, n-propyl, isopropyl, n-butyl, t-butyl, n-hexyl, etc. The same applies to the alkyl groups in O-alkyl. Alkenyl represents straight-chain or branched alkenyl groups which have preferably 3-8 carbon atoms, especially 3-6 carbon atoms. A preferred alkenyl group is the allyl group. Cycloalkyl is especially C₁-C₆-cycloalkyl, particular preference being given to cyclopentyl and cyclohexyl. Aryl (including in aralkyl) is preferably phenyl or naphthyl. When the aryl radical is a phenyl group and is substituted, it preferably has two substituents. These are present especially in the 2 and/or 4 position.

Halogen is F, Cl, Br and I, preferably Cl and Br.

M is preferably an ammonium ion, alkali metal ion or one equivalent of an alkaline earth metal or zinc ion. Suitable alkali metal ions are especially sodium and potassium ions; suitable alkaline earth metal ions are in particular magnesium, strontium and calcium ions.

R¹ is preferably a hydroxyl or amino group.

R² is preferably a hydrogen atom or an alkyl or aryl group which may be substituted as above. It preferably has one or two hydroxyl and/or alkoxy substituents.

R³ is preferably either COOM or COOR⁴ (M and R⁴ are each defined as specified above) or, when R¹ is aryl which may be substituted as specified above, is also a hydrogen atom.

In a preferred embodiment, the superabsorbent comprises compounds of the above formula in which M is an alkali metal ion or one equivalent of an alkaline earth metal or zinc ion; R¹ is a hydroxyl or amino group; R² is H or alkyl and R³ is COOM or COOR⁴, where, when R³ is COOM, M in this COOM radical is H, an alkali metal ion or one equivalent of an alkaline earth metal ion, and, when R³ is COOR⁴, R⁴ is C₁-C₆-alkyl.

In a further preferred embodiment, the superabsorbent comprises compounds of the above formula in which M is an alkali metal ion or one equivalent of an alkaline earth metal or zinc ion; R¹ is a hydroxyl or amino group; R² is aryl which is optionally substituted as specified above, especially hydroxyphenyl or C₁-C₄-alkoxyphenyl; and R³ is a hydrogen atom.

Groups 1 (H, Li, Na, K, Rb, Cs, Fr), 2 (Be, Mg, Ca, Sr, Ba, Ra), 8 (Fe, Ru, Os), 9 (Co, Rh, Ir), 10 (Ni, Pd, Pt), 12 (Zn, Cd, Hg) and 14 (C, Si, Ge, Sn, Pb) of the Periodic Table of the Elements in the current IUPAC numbering (International Union of Pure and Applied Chemistry, 104 T. W. Alexander Drive, Building 19, Research Triangle Park, N.C. 27709, U.S.A., www.iupac.org), the international organization responsible for nomenclature in the field of chemistry, correspond to groups Ia, IIa, IIb, IVa and VIIIb in the numbering used by CAS (Chemical Abstracts Service, 2540 Olentangy River Road, Columbus, Ohio 43202, U.S.A., www.cas.org).

The sulfinic acid derivatives of the above formula can be used in pure form, but optionally also in the mixture with the sulfite of the corresponding metal ion and of the corresponding sulfonic acid which results in a customary manner from the preparation of such compounds. The preparation of such sulfinic acid derivatives of the above formula is known and is described, for example, in WO 99/18 067 A1. They are also conventional commercial products and are available, for example, in the form of mixtures of the sodium salt of 2-hydroxy-2-sulfinatoacetic acid, the disodium salt of 2-hydroxy-2-sulfonatoacetic acid and sodium bisulfite from L. Brüggemann K G (Salzstrasse 131, 74076 Heilbronn, Germany, www.brueggemann.com) under the names BRÜGGOLIT® FF6M or BRÜGGOLIT® FF7, or alternatively BRUGGOLITE® FF6M or BRUGGOLITE® FF7.

To prepare the inventive superabsorbent, a superabsorbent known per se is mixed with a compound of the formula (I), a salt thereof or a mixture of compounds of the formula (I) and/or salts thereof. For the sake of simplicity, reference will be made hereinafter only to “compound of the formula (I)” or to “sulfinic acid derivative”, but this is not intended to exclude salts of such compounds and mixtures of such compounds and/or salts thereof, but merely to avoid always mentioning them too.

In general, the inventive superabsorbent comprises at least 0.0001% by weight of sulfinic acid derivative, preferably at least 0.001% by weight and more preferably at least 0.025% by weight, for example at least 0.05% by weight or at least 0.1% by weight, and generally at most 3% by weight, preferably at most 2% by weight and more preferably at most 0.5% by weight, for example at most 0.35% by weight or 0.2% by weight, based in each case on the total weight of the inventive superabsorbent.

The mixing of superabsorbents known per se with a compound of the formula (I) can be effected by any known mixing process. The compound of the formula (I) can be mixed in in bulk, as a solution or as a suspension in a solvent or suspension medium; owing to the easier homogeneous distribution, it is preferably mixed in as a solution or suspension. This does not necessarily produce a physical mixture of superabsorbent known per se and sulfinic acid derivative of the formula (I) which can be separated simply by mechanical measures. The sulfinic acid derivative may quite possibly enter into a more definite bond with the superabsorbent, for example in the form of a comparatively firmly adhering surface layer or in the form of particles of the sulfinic acid derivative adhering firmly to the surface of the superabsorbent particles. The mixing of the sulfinic acid derivative into the superabsorbents known per se can quite possibly also be understood and referred to as “coating”.

If the superabsorbent known per se is mixed with a solution or suspension of the sulfinic acid derivative, the solvent or suspension medium used is a solvent or suspension medium which is chemically compatible both with the superabsorbent and with the sulfinic acid derivative, i.e. does not enter into any undesired chemical reactions therewith. Typically, water or an organic solvent is used, for example an alcohol or polyol, or mixtures thereof. Examples of suitable solvents or suspension media are water, isopropanol/water, 1,3-propanediol/water and propylene glycol/water, where the mass mixing ratio is preferably from 20:80 to 40:60. A surfactant can be added to the solution or suspension.

The sulfinic acid derivative is generally mixed with the superabsorbent known per se in exactly the same way as the solution or suspension which comprises a surface postcrosslinker and is applied to the superabsorbent for surface postcrosslinking, as described below. The sulfinic acid derivative can be applied as a constituent of the solution applied for surface postcrosslinking or of one of the components thereof to an (as yet) nonpostcrosslinked superabsorbent (a “base polymer”), i.e. the sulfinic acid derivative is added to the solution of the surface postcrosslinker or to one of the components thereof. The superabsorbent coated with surface postcrosslinker and sulfinic acid derivative then passes through the further process steps required for surface postcrosslinking, for example a thermally induced reaction of the surface postcrosslinker with the superabsorbent. This process is comparatively simple and economically viable.

If ultrahigh stability to discoloration is essential, the sulfinic acid derivative is preferably applied in a dedicated process step after the surface postcrosslinking. If the sulfinic acid derivative is applied in the form of a solution or suspension, the application to the already surface postcrosslinked superabsorbent is effected in the same way as the application of the surface postcrosslinker to the base polymer. Usually, but not necessarily, this is followed, just like in the surface postcrosslinking step, by heating, in order to dry the superabsorbent again. The temperature established in this drying step is then, however, generally at most 110° C., preferably at most 100° C. and more preferably at most 90° C., in order to prevent undesired reactions of the sulfinic acid derivative. The temperature is adjusted such that, in view of the residence time in the drying unit, the desired water content of the superabsorbent is achieved. It is also entirely possible and convenient to add the sulfinic acid derivative individually or together with other customary assistants, for example antidusting agents, anticaking agents or water to remoisten the superabsorbent, as described below for these assistants, for example in a cooler connected downstream of the surface postcrosslinking step. The temperature of the polymer particles in this case is between 0° C. and 190° C., preferably less than 160° C., more preferably less than 130° C., even more preferably less than 100° C. and most preferably less than 70° C. The polymer particles are optionally cooled rapidly after coating to temperatures below the decomposition temperature of the sulfinic acid derivative.

The superabsorbent known per se, which becomes the inventive superabsorbent by virtue of the sulfinic acid derivative being mixed in, may be any superabsorbent. In general, such superabsorbents are crosslinked hydrophilic polymers, especially polymers of (co)polymerized hydrophilic monomers, graft (co)polymers of one or more hydrophilic monomers on a suitable graft base, crosslinked cellulose or starch ethers, crosslinked carboxymethylcellulose, partially crosslinked polyalkylene oxide or natural products which are swellable in aqueous liquids, for example guar derivatives. Preference is given to using a superabsorbent based on partially neutralized acrylic acid. Superabsorbents are characterized in particular by their ability to absorb and retain fluids. The superabsorbents to be coated with sulfinic acid derivative in accordance with the invention are selected such that the finished superabsorbent has a centrifuge retention capacity (CRC, see below for test method) of at least 5 g/g, preferably of at least 10 g/g and more preferably of at least 20 g/g. Further suitable minimum values of the CRC are, for example, 25 g/g, 30 g/g or 35 g/g. It is typically not more than 40 g/g. The CRCs of many superabsorbents which are currently available on the market and can be used in accordance with the invention are in the range from 28 to 33 g/g.

The superabsorbents to be coated in accordance with the invention with sulfinic acid derivative are additionally selected such that the finished superabsorbent typically has an absorbency under load (AUL0.7.psi, see below for test method) of at least 18 g/g, preferably at least 20 g/g, preferentially at least 22 g/g, more preferably at least 23 g/g, even more preferably at least 24 g/g and typically not more than 30 g/g.

The superabsorbents to be coated in accordance with the invention with sulfinic acid derivative are additionally selected such that the finished superabsorbent typically has a saline flow conductivity (SFC, see below for test method) of at least 10×10⁻⁷ cm³ s/g, preferably at least 30×10⁻⁷ cm³ s/g, preferentially at least 50×10⁻⁷ cm³ s/g, more preferably at least 80×10⁻⁷ cm³ s/g, even more preferably at least 100×10⁻⁷ cm³ s/g and typically not more than 1000×10⁻⁷ cm³ s/g.

CRC, AUL and SFC of the superabsorbent are generally not influenced significantly by the coating of the superabsorbent with the sulfinic acid derivative. These parameters are adjusted in a manner which is known and customary in the production of superabsorbents. When the sulfinic acid derivative is added in the course of surface postcrosslinking of a base polymer or simultaneously with other additives which influence these properties of superabsorbents, the adjustment of these parameters of course takes place during the coating of the superabsorbent with sulfinic acid derivative. However, this is determined by these other measures and not by the presence of the sulfinic acid derivative.

A superabsorbent to be coated in accordance with the invention with sulfinic acid derivative is, for example, prepared by aqueous solution polymerization of a monomer mixture comprising

a) at least one ethylenically unsaturated monomer which bears acid groups and is optionally present at least partly in salt form,

b) at least one crosslinker,

c) at least one initiator,

d) optionally one or more ethylenically unsaturated monomers copolymerizable with the monomers specified under a), and

e) optionally one or more water-soluble polymers.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids or salts thereof, such as acrylic acid, methacrylic acid, maleic acid or salts thereof, maleic anhydride and itaconic acid or salts thereof. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerization. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, acrylic acid purified according to WO 2004/035514 A1 comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers a) is preferably at least 50 mol %, more preferably at least 90 mol %, most preferably at least 95 mol %.

The monomer solution comprises preferably at most 250 ppm by weight, preferably at most 130 ppm by weight, more preferably at most 70 ppm by weight and preferably at least 10 ppm by weight, more preferably at least 30 ppm by weight, especially around 50 ppm by weight, of hydroquinone monoether, based in each case on the unneutralized monomer a); neutralized monomer a), i.e. a salt of the monomer a), is considered for arithmetic purposes as unneutralized monomer. For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerizable groups which can be polymerized free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 530 438 A1, di- and triacrylates, as described in EP 547 847 A1, EP 559 476 A1, EP 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/32962 A2.

Preferred crosslinkers b) are pentaerythrityl triallyl ether, tetraallyloxyethane, methylenebismethacrylamide, 15- to 20-tuply ethoxylated trimethylolpropane triacrylate, 15- to 20-tuply ethoxylated glyceryl triacrylate, polyethylene glycol diacrylate with between 4 and 45 —CH₂CH₂O— units in the molecule chain, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably from 0.05 to 1.5% by weight, more preferably from 0.1 to 1% by weight, most preferably from 0.3 to 0.6% by weight, based in each case on monomer a). With rising crosslinker content, the centrifuge retention capacity (CRC) falls and the absorption under a pressure of 0.3 psi (AUL0.3 psi) rises.

The initiators c) may be all compounds which generate free radicals under the polymerization conditions, for example thermal initiators, redox initiators, photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably the above-described mixture of the sodium salt of 2-hydroxy-2-sulfinatoacetic acid, the disodium salt of 2-hydroxy-2-sulfonatoacetic acid and sodium bisulfite (Brüggolit® FF6M or Brüggolit® FF7).

Ethylenically unsaturated monomers d) copolymerizable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate, maleic acid or salts thereof, and maleic anhydride.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight, most preferably from 50 to 65% by weight. It is also possible to use monomer suspensions, i.e. oversaturated monomer solutions. With rising water content, the energy requirement in the subsequent drying rises, and, with falling water content, the heat of polymerization can only be removed inadequately.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

The monomer mixture may comprise further components. Examples of further components used in monomer mixtures of this kind are, for instance, chelating agents, in order to keep metal ions in solution.

Suitable polymerization reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/38402 A1. Polymerization on a belt is described, for example, in DE 38 25 366 A1 and U.S. Pat. No. 6,241,928. Polymerization in a belt reactor forms a polymer gel, which has to be comminuted in a further process step, for example in a meat grinder, extruder or kneader. However, it is also possible to produce spherical or differently shaped superabsorbent particles by suspension, spray or droplet polymerization processes.

The acid groups of the resulting polymer gels have typically been partially neutralized. Neutralization is preferably carried out at the monomer stage; in other words, salts of the monomers bearing acid groups or, to be precise, a mixture of monomers bearing acid groups and salts of the monomers bearing acid groups (“partly neutralized acid”) are used as component a) in the polymerization. This is typically done by mixing the neutralizing agent as an aqueous solution or preferably also as a solid into the monomer mixture intended for polymerization or preferably into the monomer bearing acid groups or a solution thereof. The degree of neutralization is preferably from 25 to 95 mol %, more preferably from 50 to 80 mol %, most preferably from 65 to 72 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof.

However, it is also possible to carry out neutralization after the polymerization, at the stage of the polymer gel formed in the polymerization. It is also possible to neutralize up to 40 mol %, preferably from 10 to 30 mol % and more preferably from 15 to 25 mol % of the acid groups before the polymerization by adding a portion of the neutralizing agent actually to the monomer solution and setting the desired final degree of neutralization only after the polymerization, at the polymer gel stage. When the polymer gel is neutralized at least partly after the polymerization, the polymer gel is preferably comminuted mechanically, for example by means of an extruder, in which case the neutralizing agent can be sprayed, sprinkled or poured on and then carefully mixed in. To this end, the gel mass obtained can be repeatedly extruded for homogenization.

However, preference is given to performing the neutralization at the monomer stage. In other words: in a very particularly preferred embodiment, the monomer a) used is a mixture of from 25 to 95 mol %, more preferably from 50 to 80 mol %, more preferably from 65 to 72 mol %, of salt of the monomer bearing acid groups, and the remainder to 100 mol % of monomer bearing acid groups. This mixture is, for example, a mixture of sodium acrylate and acrylic acid or a mixture of potassium acrylate and acrylic acid.

In a preferred embodiment, the neutralizing agent used for the neutralization is one whose iron content is generally below 10 ppm by weight, preferably below 2 ppm by weight and more preferably below 1 ppm by weight. Likewise desired is a low content of chloride and anions of oxygen acids of chlorine. A suitable neutralizing agent is, for example, the 50% by weight sodium hydroxide solution or potassium hydroxide solution which is typically traded as “membrane grade”; even more pure and likewise suitable, but also more expensive, is the 50% by weight sodium hydroxide solution or potassium hydroxide solution typically traded as “amalgam grade” or “mercury process”.

The polymer gel obtained from the aqueous solution polymerization and optional subsequent neutralization is then preferably dried with a belt drier until the residual moisture content is preferably from 0.5 to 15% by weight, more preferably from 1 to 10% by weight, most preferably from 2 to 8% by weight (see below for test method for the residual moisture or water content). In the case of too high a residual moisture content, the dried polymer gel has too low a glass transition temperature Tg and can be processed further only with difficulty. In the case of too low a residual moisture content, the dried polymer gel is too brittle and, in the subsequent comminution steps, undesirably large amounts of polymer particles with too low a particle size (“fines”) are obtained. The solids content of the gel before drying is generally from 25 to 90% by weight, preferably from 30 to 80% by weight, more preferably from 35 to 70% by weight, most preferably from 40 to 60% by weight. Optionally, however, it is also possible to dry using a fluidized bed drier or a heatable mixer with a mechanical mixing unit, for example a paddle drier or a similar drier with mixing tools of different design. Optionally, the drier can be operated under nitrogen or another nonoxidizing inert gas or at least under reduced partial oxygen pressure in order to prevent oxidative yellowing processes. In general, however, even sufficient venting and removal of water vapor leads to an acceptable product. A very short drying time is generally advantageous with regard to color and product quality.

During the drying, the residual monomer content in the polymer particles is also reduced, and last residues of the initiator are destroyed.

Thereafter, the dried polymer gel is ground and classified, apparatus usable for the grinding typically including single- or multistage roll mills, preferably two- or three-stage roll mills, pin mills, hammer mills or vibratory mills. Oversize gel lumps which often still have not dried on the inside are elastomeric, lead to problems in the grinding and are preferably removed before the grinding, which can be done in a simple manner by wind sifting or by means of a screen (“protective screen” for the mill). In view of the mill used, the mesh size of the screen should be selected such that a minimum level of disruption resulting from oversize, elastomeric particles occurs.

Excessively large, insufficiently finely ground superabsorbent particles are perceptible as coarse particles in their predominant use, in hygiene products such as diapers; they also lower the mean initial swell rate of the superabsorbent. Both are undesired. Advantageously, coarse-grain polymer particles are therefore removed from the product. This is typically done by classification processes, for example wind sifting, or by screening through a screen with a mesh size of at most 1000 μm, preferably at most 900 μm, more preferably at most 850 μm and most preferably at most 800 μm. For example, screens of mesh size 700 μm, 650 μm or 600 μm are used. The coarse polymer particles (“oversize”) removed may, for cost optimization, be sent back to the grinding and screening cycle or be processed further separately.

Polymer particles with too low a particle size lower the permeability (SFC). Advantageously, fine polymer particles are therefore also removed in this classification. This can, if screening is effected, conveniently be used through a screen of mesh size at most 300 μm, preferably at most 200 μm, more preferably at most 150 μm and most preferably at most 100 μm. The fine polymer particles (“undersize” or “fines”) removed can, for cost optimization, be sent back as desired to the monomer stream, to the polymerizing gel or to the fully polymerized gel before the drying of the gel.

The mean particle size of the polymer particles removed as the product fraction is generally at least 200 μm, preferably at least 250 μm and more preferably at least 300 μm, and generally at most 600 μm and more preferably at most 500 μm. The proportion of particles with a particle size of at least 150 μm is generally at least 90% by weight, more preferably at least 95% by weight and most preferably at least 98% by weight. The proportion of particles with a particle size of at most 850 μm is generally at least 90% by weight, more preferably at least 95% by weight and most preferably at least 98% by weight.

The polymer thus prepared has superabsorbent properties and is covered by the term “superabsorbent”. Its CRC is typically comparatively high, but its AUL or SFC comparatively low. A surface nonpostcrosslinked superabsorbent of this type is often referred to as “base polymer” to distinguish it from a surface postcrosslinked superabsorbent produced therefrom. In the context of the present invention, a base polymer can quite possibly be processed to an inventive superabsorbent by addition of sulfinic acid derivative even without surface postcrosslinking.

To further improve the properties, especially increase the AUL and SFC values (which lowers the CRC value), the superabsorbent particles can be surface postcrosslinked. Suitable postcrosslinkers are compounds which comprise groups which can form bonds with at least two functional groups of the superabsorbent particles. In the case of the acrylic acid/sodium acrylate-based superabsorbents prevalent on the market, suitable surface postcrosslinkers are compounds which comprise groups which can form bonds with at least two carboxylate groups. Preferred postcrosslinkers are amide acetals or carbamates of the general formula (II)

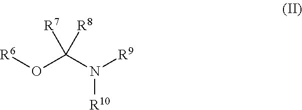

in which

R⁶ is C₁-C₁₂-alkyl, C₂-C₁₂-hydroxyalkyl, C₂-C₁₂-alkenyl or C₆-C₁₂-aryl,

R⁷ is X or OR¹¹,

R⁸ is hydrogen, C₁-C₁₂-alkyl, C₂-C₁₂-hydroxyalkyl, C₂-C₁₂-alkenyl or C₆-C₁₂-aryl, or X,

R⁹ is C₁-C₁₂-alkyl, C₂-C₁₂-hydroxyalkyl, C₂-C₁₂-alkenyl or C₆-C₁₂-aryl,

R¹⁰ is hydrogen, C₁-C₁₂-alkyl, C₂-C₁₂-hydroxyalkyl, C₂-C₁₂-alkenyl, C₁-C₁₂-acyl or C₆-C₁₂-aryl,

R11 is C₁-C₁₂-alkyl, C₂-C₁₂-hydroxyalkyl, C₂-C₁₂-alkenyl or C₆-C₁₂-aryl and

X is a carbonyl oxygen for the R⁷ and R⁸ radicals together,

where R⁶ and R⁹ and/or R¹⁰ and R¹¹ may be a bridged C₂-C₆-alkanediyl and where the abovementioned R⁶ to R¹¹ radicals may also have a total of from one to two free valences and may be joined to at least one suitable base structure by these free valances,

or polyhydric alcohols, the polyhydric alcohol preferably having a molecular weight of less than 100 g/mol, preferably of less than 90 g/mol, more preferably of less than 80 g/mol, most preferably of less than 70 g/mol, per hydroxyl group, and no vicinal, geminal, secondary or tertiary hydroxyl groups, and polyhydric alcohols are either diols of the general formula (IIa)

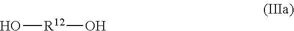

in which R¹² is either an unbranched dialkyl radical of the formula —(CH₂)ₙ— where n is an integer from 3 to 20, preferably from 3 to 12, and both hydroxyl groups are terminal, or R¹² is an unbranched, branched or cyclic dialkyl radical, or polyols of the general formula (IIb)

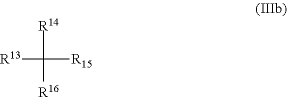

in which the R¹³, R¹⁴, R¹⁵, R¹⁶ radicals are each independently hydrogen, hydroxyl, hydroxymethyl, hydroxyethyloxymethyl, 1-hydroxyprop-2-yloxymethyl, 2-hydroxypropyloxymethyl, methyl, ethyl, n-propyl, isopropyl, n-butyl, n-pentyl, n-hexyl, 1,2-dihydroxyethyl, 2-hydroxyethyl, 3-hydroxypropyl or 4-hydroxybutyl, and a total of 2, 3 or 4, preferably 2 or 3, hydroxyl groups are present, and not more than one of the R¹³, R¹⁴, R¹⁵, and R¹⁶ radicals is hydroxyl,

or cyclic carbonates of the general formula (IV)

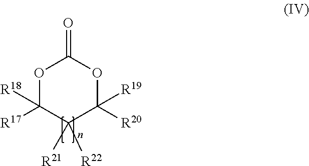

in which R₁₇, R₁₈, R₁₉, R₂₀, R₂₁ and R₂₂ are each independently hydrogen, methyl, ethyl, n-propyl, isopropyl, n-butyl, sec-butyl or isobutyl, and n is either 0 or 1,

or bisoxazolines of the general formula (V)

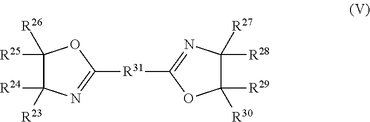

in which R₂₃, R₂₄, R₂₅, R₂₆, R₂₇, R₂₈, R₂₉ and R₃₀ are each independently hydrogen, methyl, ethyl, n-propyl, isopropyl, n-butyl, sec-butyl or isobutyl, and R³¹ is a single bond, a linear, branched or cyclic C₂-C₁₂-dialkyl radical, or a polyalkoxydiyl radical which is formed from one to ten ethylene oxide and/or propylene oxide units, as possessed, for example, by polyglycoldicarboxylic acids.

Further suitable postcrosslinkers are (VI) β-hydroxyalkylamides (for example Primid® XL-512 which is sold by EMS-Chemie, Reichenauerstrasse, 7013 Domat/Ems, Switzerland), (VII) polyepoxides, (VIII) polyaziridenes and (IX) oxetane derivatives.

Preferred postcrosslinkers of the general formula (II) are 2-oxazolidones such as 2-oxazolidone and N-(2-hydroxyethyl)-2-oxazolidone, N-methyl-2-oxazolidone, N-acyl-2-oxazolidones such as N-acetyl-2-oxazolidone, 2-oxotetrahydro-1,3-oxazine, bicyclic amide acetals such as 5-methyl-1-aza-4,6-dioxabicyclo[3.3.0]octane, 1-aza-4,6-dioxabicyclo[3.3.0]octane and 5-isopropyl-1-aza-4,6-dioxabicyclo[3.3.0]octane, bis-2-oxazolidones and poly-2-oxazolidones.

Particularly preferred postcrosslinkers of the general formula (II) are 2-oxazolidone, N-methyl-2-oxazolidone, N-(2-hydroxyethyl)-2-oxazolidone and N-hydroxypropyl-2-oxazolidone.

Preferred postcrosslinkers of the general formula (IIIa) are 1,3-propanediol, 1,5-pentanediol, 1,6-hexanediol and 1,7-heptanediol. Further examples of postcrosslinkers of the formula (IIa) are 1,3-butanediol, 1,8-octanediol, 1,9-nonanediol and 1,10-decanediol.

The diols are preferably water-soluble, the diols of the general formula (IIIa) being water-soluble at 23° C. to an extent of at least 30% by weight, preferably to an extent of at least 40% by weight, more preferably to an extent of at least 50% by weight, most preferably at least to an extent of 60% by weight, for example 1,3-propanediol and 1,7-heptanediol. Even more preferred are those postcrosslinkers which are liquid at 25° C.

Preferred postcrosslinkers of the general formula (IIIb) are butane-1,2,3-triol, butane-1,2,4-triol, glycerol, trimethylolpropane, trimethylolethane, pentaerythritol, 1- to 3-tuply (per molecule) ethoxylatecl glycerol, trimethylolethane or trimethylolpropane and 1- to 3-tuply (per molecule) propoxylated glycerol, trimethylolethane or trimethylolpropane. Additionally preferred are 2-tuply ethoxylated or propoxylated neopentyl glycol. Particular preference is given to 2-tuply and 3-tuply ethoxylated glycerol, neopentyl glycol, 2-methyl-1,3-propanediol and trimethylolpropane.

Preferred polyhydric alcohols (IIIa) and (IIIb) have, at 23° C., a viscosity of less than 3000 mPas, preferably less than 1500 mPas, preferentially less than 1000 mPas, more preferably less than 500 mPas, most preferably less than 300 mPas.

Particularly preferred postcrosslinkers of the general formula (IV) are ethylene carbonate and propylene carbonate.

A particularly preferred postcrosslinker of the general formula (V) is 2,2′-bis(2-oxazoline).

The preferred postcrosslinkers minimize side reactions and subsequent reactions which lead to volatile and hence malodorous compounds. The water-absorbent polymers prepared with the preferred postcrosslinkers are therefore odor-neutral even in the moistened state. Moreover, the preferred postcrosslinkers are toxicologically safe to an exceptional degree.

It is possible to use an individual postcrosslinker from the above selection or any mixtures of different postcrosslinkers.

The postcrosslinker is generally used in an amount of at least 0.001% by weight, preferably of at least 0.02% by weight, more preferably of at least 0.05% by weight, and generally at most 2% by weight, preferably at most 1% by weight, more preferably at most 0.3% by weight, for example at most 0.15% by weight or at most 0.095% by weight, based in each case on the mass of the base polymer.

The postcrosslinking is typically carried out in such a way that a solution of the postcrosslinker is sprayed onto the dried base polymer particles. After the spray application, the polymer particles coated with postcrosslinker are dried thermally, and the postcrosslinking reaction may take place either before or during the drying. If surface postcrosslinkers with polymerizable groups are used, the surface postcrosslinking can also be effected by means of free-radically induced polymerization of such groups by means of common free-radical formers or else by means of high-energy radiation, for example UV light. This can be done in parallel or instead of the use of postcrosslinkers which form covalent or ionic bonds to functional groups at the surface of the base polymer particles.

The spray application of the postcrosslinker solution is preferably carried out in mixers with moving mixing tools, such as screw mixers, disk mixers or paddle mixers, or mixers with other mixing tools. Particular preference is given, however, to vertical mixers. However, it is also possible to spray on the postcrosslinker solution in a fluidized bed. Suitable mixers are, for example, obtainable as Pflugschar® plowshare mixers from Gebr. Lödige Maschinenbau GmbH, Elsener-Strasse 7-9, 33102 Paderborn, Germany, or as Schugi® Flexomix® mixers, Vrieco-Nauta® mixers or Turbulizer® mixers from Hosokawa Micron BV, Gildenstraat 26, 7000 AB Doetinchem, the Netherlands.

The spray nozzles usable are not subject to any restriction. Suitable nozzles and atomization systems are described, for example, in the following references: Zerstäuben von Flüssigkeiten [Atomization of Liquids], Expert-Verlag, vol. 660, Reihe Kontakt & Studium, Thomas Richter (2004), and in Zerstäubungstechnik [Atomization Technology], Springer-Verlag, VDI-Reihe, Günter Wozniak (2002). It is possible to use mono- and polydisperse spray systems. Among the polydisperse systems, one-substance pressurized nozzles (jet- or lamellar-forming), rotational atomizers, two-substance atomizers, ultrasound atomizers and impingement nozzles are suitable. In the case of the two-substance atomizers, the liquid phase can be mixed with the gas phase either internally or externally. The spray profile of the nozzles is uncritical and may assume any desired form, for example a round jet, flat jet, wide angle round beam or circular ring spray profile. It is advantageous to use a nonoxidizing gas if two-substance atomizers are used, particular preference being given to nitrogen, argon or carbon dioxide. The liquid to be sprayed can be supplied to such nozzles under pressure. The liquid to be sprayed can be atomized by decompressing it in the die bore on attainment of a particular minimum velocity. In addition, it is also possible to use one-substance nozzles for the inventive purpose, for example slot dies or impingement chambers (full-cone nozzles) (for example from Düsen-Schlick GmbH, Germany, or from Spraying Systems Deutschland GmbH, Germany). Such nozzles are also described in EP 0 534 228 A1 and EP 1 191 051 A2.

The postcrosslinkers are typically used in the form of an aqueous solution. When exclusively water is used as the solvent, a surfactant or deagglomeration assistant is advantageously added to the postcrosslinker solution or actually to the base polymer. This improves the wetting performance and reduces the tendency to form lumps.

All anionic, cationic, nonionic and amphoteric surfactants are suitable as deagglomeration assistants, but preference is given to nonionic and amphoteric surfactants for skin compatible reasons. The surfactant may also comprise nitrogen. For example, sorbitan monoesters, such as sorbitan monococoate and sorbitan monolaurate, or ethoxylated variants thereof, for example Polysorbat 20®, are added. Further suitable deagglomeration assistants are the ethoxylated and alkoxylated derivatives of 2-propylheptanol, which are sold under the Lutensol XL® and Lutensol XP® brands (BASF SE, Carl-Bosch-Strasse 38, 67056 Ludwigshafen, Germany).

The deagglomeration assistant can be metered in separately or added to the postcrosslinker solution. Preference is given to simply adding the deagglomeration assistant to the postcrosslinker solution.

The amount of the deagglomeration assistant used, based on base polymer, is, for example, from 0 to 0.1% by weight, preferably from 0 to 0.01% by weight, more preferably from 0 to 0.002% by weight. The deagglomeration assistant is preferably metered in such that the surface tension of an aqueous extract of the swollen base polymer and/or of the swollen postcrosslinked water-absorbing polymer at 23° C. is at least 0.060 N/m, preferably at least 0.062 N/m, more preferably at least 0.065 N/m, and advantageously at most 0.072 N/m.

The aqueous postcrosslinker solution may, as well as the at least one postcrosslinker, also comprise a cosolvent. The content of nonaqueous solvent or total amount of solvent can be used to adjust the penetration depth of the postcrosslinker into the polymer particles. Industrially readily available cosolvents are C1-C6-alkohols such as methanol, ethanol, n-propanol, isopropanol, n-butanol, sec-butanol, tert-butanol or 2-methyl-1-propanol, C2-C5-diols such as ethylene glycol, 1,2-propylene glycol or 1,4-butanediol, ketones such as acetone, or carboxylic esters such as ethyl acetate. A disadvantage of some of these cosolvents is that they have typical intrinsic odors.

The cosolvent itself is ideally not a postcrosslinker under the reaction conditions. However, it may arise in the boundary case and depending on the residence time and temperature that the cosolvent contributes partly to crosslinking. This is the case especially when the postcrosslinker is relatively sluggish and therefore can also be its own cosolvent, as, for example, in the case of use of cyclic carbonates of the general formula (IV), diols of the general formula (IIIa) or polyols of the general formula (IIIb). Such postcrosslinkers can be used in a mixture with more reactive postcrosslinkers or else in the function as a cosolvent, since the actual postcrosslinking reaction can then be carried out at lower temperatures and/or with shorter residence times than in the absence of the more reactive crosslinker. Since the cosolvent is used in relatively large amounts and also remains partly in the product, it must not be toxic.

Also suitable as cosolvents in the process according to the invention are the diols of the general formula (IIIa), the polyols of the general formula (IIIb), and the cyclic carbonates of the general formula (IV). They fulfil this function in the presence of a reactive postcrosslinker of the general formula (II) and/or (V) and/or of a di- or triglycidyl compound. Preferred cosolvents in the process according to the invention are, however, especially the diols of the general formula (IIIa), especially when a reaction of the hydroxyl groups is hindered sterically by neighboring groups. Although such diols are also suitable in principle as postcrosslinkers, this requires significantly higher reaction temperatures or optionally higher use amounts than for sterically unhindered diols.

Particularly preferred combinations of low-reactivity postcrosslinker as a cosolvent and reactive postcrosslinker are combinations of preferred polyhydric alcohols, diols of the general formula (IIIa) and polyols of the general formula (IIIb), with amide acetals or carbamates of the general formula (II).

Suitable combinations are, for example, 2-oxazolidone/1,2-propanediol and N-(2-hydroxyethyl)-2-oxazolidone/1,2-propanediol, and also ethylene glycol diglycidyl ether/1,2-propanediol.

Very particularly preferred combinations are 2-oxazolidone/1,3-propanediol and N-(2-hydroxyethyl)-2-oxazolidone/1,3-propanediol.

Further preferred combinations are those with ethylene glycol diglycidyl ether or glyceryl di- or triglycidyl ether with the following solvents, cosolvents or cocrosslinkers: isopropanol, 1,3-propanediol, 1,2-propylene glycol or mixtures thereof.

Further preferred combinations are those with 2-oxazolidone or (2-hydroxyethyl)-2-oxazolidone in the following solvents, cosolvents or cocrosslinkers: isopropanol, 1,3-propanediol, 1,2-propylene glycol, ethylene carbonate, propylene carbonate or mixtures thereof.

Frequently, the concentration of the cosolvent in the aqueous postcrosslinker solution is from 15 to 50% by weight, preferably from 15 to 40% by weight, more preferably from 20 to 35% by weight, based on the postcrosslinker solution. In the case of cosolvents of only limited water miscibility, the aqueous postcrosslinker solution will advantageously be adjusted such that only one phase is present, optionally by lowering the concentration of the cosolvent.

In a preferred embodiment, no cosolvent is used. The postcrosslinker is then employed only as a solution in water, optionally with addition of a deagglomeration assistant.

The concentration of the at least one postcrosslinker in the aqueous postcrosslinker solution is typically from 1 to 20% by weight, preferably from 1.5 to 10% by weight, more preferably from 2 to 5% by weight, based on the postcrosslinker solution.

The total amount of the postcrosslinker solution based on base polymer is typically from 0.3 to 15% by weight, preferably from 2 to 6% by weight.

The actual surface postcrosslinking by reaction of the surface postcrosslinker with functional groups at the surface of the base polymer particles is usually carried out by heating the base polymer wetted with surface postcrosslinker solution, typically referred to as “drying” (but not to be confused with the above-described drying of the polymer gel from the polymerization, in which typically very much more liquid has to be removed). The drying can be effected in the mixer itself, by heating the jacket, by means of heat exchange surfaces or by blowing in warm gases. Simultaneous admixing of the superabsorbent with surface postcrosslinker and drying can be effected, for example, in a fluidized bed drier. The drying is, however, usually carried out in a downstream drier, for example a tray drier, a rotary tube oven, a paddle or disk drier or a heatable screw. Suitable driers are, for example, obtainable as Solidair® or Torusdisc® driers from Bepex International LLC, 333 NE. Taft Street, Minneapolis, Minn. 55413, U.S.A., or as paddle driers or else as fluidized bed driers from Nara Machinery Co., Ltd., European Branch, Europaallee 46, 50226 Frechen, Germany.

It is possible to heat the polymer particles by means of contact surfaces in a downstream drier for the purpose of drying and performing the surface postcrosslinking, or by means of warm inert gas supply, or by means of a mixture of one or more inert gases with steam, or only with steam alone. In the case of supply of the heat by means of contact surfaces, it is possible to perform the reaction under inert gas at slightly or completely reduced pressure. In the case of use of steam for direct heating of the polymer particles, it is desirable in accordance with the invention to operate the drier under standard pressure or elevated pressure. In this case, it may be advisable to split up the postcrosslinking step into a heating step with steam and a reaction step under inert gas but without steam. This can be achieved in one or more apparatuses. According to the invention, the polymer particles can be heated with steam as early as in the postcrosslinking mixer. The base polymer used may still have a temperature of from 10 to 120° C. from preceding process steps; the postcrosslinker solution may have a temperature of from 0 to 70° C. In particular, the postcrosslinker solution can be heated to reduce the viscosity.

Preferred drying temperatures are in the range from 100 to 250° C., preferably from 120 to 220° C., more preferably from 130 to 210° C., most preferably from 150 to 200° C. The preferred residence time at this temperature in the reaction mixer or drier is preferably at least 10 minutes, more preferably at least 20 minutes, most preferably at least 30 minutes, and typically at most 60 minutes. Typically, the drying is conducted such that the superabsorbent has a residual moisture content of generally at least 0.1% by weight, preferably at least 0.2% by weight and most preferably at least 0.5% by weight, and generally at most 15% by weight, preferably at most 10% by weight and more preferably at most 8% by weight.

The postcrosslinking may take place under standard atmospheric conditions. “Standard atmospheric conditions” means that no technical precautions are taken in order to reduce the partial pressure of oxidizing gases, such as that of atmospheric oxygen, in the apparatus in which the postcrosslinking reaction predominantly takes place (the “postcrosslinking reactor”, typically the drier). However, preference is given to performing the postcrosslinking reaction under reduced partial pressure of oxidizing gases. Oxidizing gases are substances which, at 23° C., have a vapor pressure of at least 1013 mbar and act as oxidizing agents in combustion processes, for example oxygen, nitrogen oxide and nitrogen dioxide, especially oxygen. The partial pressure of oxidizing gases is preferably less than 140 mbar, preferably less than 100 mbar, more preferably less than 50 mbar, most preferably less than 10 mbar. When the thermal postcrosslinking is carried out at ambient pressure, i.e. at a total pressure around 1013 mbar, the total partial pressure of the oxidizing gases is determined by their proportion by volume. The proportion of the oxidizing gases is preferably less than 14% by volume, preferably less than 10% by volume, more preferably less than 5% by volume, most preferably less than 1% by volume.

The postcrosslinking can be carried out under reduced pressure, i.e. at a total pressure of less than 1013 mbar. The total pressure is typically less than 670 mbar, preferably less than 480 mbar, more preferably less than 300 mbar, most preferably less than 200 mbar. When drying and postcrosslinking are carried out under air with an oxygen content of 20.8% by volume, the partial oxygen pressures corresponding to the abovementioned total pressures are 139 mbar (670 mbar), 100 mbar (480 mbar), 62 mbar (300 mbar) and 42 mbar (200 mbar), the particular total pressures being in the brackets. Another means of lowering the partial pressure of oxidizing gases is the introduction of nonoxidizing gases, especially inert gases, into the apparatus used for postcrosslinking. Suitable inert gases are substances which are present in gaseous form in the postcrosslinking drier at the postcrosslinking temperature and the given pressure and do not have an oxidizing action on the constituents of the drying polymer particles under these conditions, for example nitrogen, carbon dioxide, argon, steam, preference being given to nitrogen. The amount of inert gas is generally from 0.0001 to 10 m³, preferably from 0.001 to 5 m³, more preferably from 0.005 to 1 m³ and most preferably from 0.005 to 0.1 m³, based on 1 kg of superabsorbent.

In the process according to the invention, the inert gas, if it does not comprise steam, can be blown into the postcrosslinking drier via nozzles; however, particular preference is given to adding the inert gas to the polymer particle stream via nozzles actually within or just upstream of the mixer, by admixing the superabsorbent with surface postcrosslinker.

It will be appreciated that vapors of cosolvents removed from the drier can be condensed again outside the drier and optionally recycled.

In a preferred embodiment of the present invention, polyvalent cations are applied to the particle surface in addition to the postcrosslinkers before, during or after the postcrosslinking. This is in principle a further surface postcrosslinking by means of ionic noncovalent bonds, but is occasionally also referred to as “complexation” with the metal ions in question or simply as “coating” with the substances in question (the “complexing agent”).

This application of polyvalent cations is effected by spray application of solutions of di- or polyvalent cations, usually di-, tri- or tetravalent metal cations, but also polyvalent cations such as polymers formed, in a formal sense, entirely or partly from vinylamine monomers, such as partly or fully hydrolyzed polyvinylamide (so-called “polyvinylamine”), whose amine groups are always—even at very high pH values—present partly in protonated form to give ammonium groups. Examples of usable divalent metal cations are especially the divalent cations of metals of groups 2 (especially Mg, Ca, Sr, Ba), 7 (especially Mn), 8 (especially Fe), 9 (especially Co), 10 (especially Ni), 11 (especially Cu) and 12 (especially Zn) of the Periodic Table of the Elements. Examples of usable trivalent metal cations are especially the trivalent cations of metals of groups 3 including the lanthanides (especially Sc, Y, La, Ce), 8 (especially Fe), 11 (especially Au) and 13 (especially Al) of the Periodic Table of the Elements. Examples of usable tetravalent cations are especially the tetravalent cations of metals from the lanthanides (especially Ce) and group 4 (especially Ti, Zr, Hf) of the Periodic Table of the Elements. The metal cations can be used either alone or in a mixture with one another. Particular preference is given to the use of trivalent metal cations. Very particular preference is given to the use of aluminum cations.

Among the metal cations mentioned, suitable metal salts are all of those which possess sufficient solubility in the solvent to be used. Particularly suitable metal salts are those with weakly complexing anions, for example chloride, nitrate and sulfate, hydrogensulfate, carbonate, hydrogencarbonate, nitrate, phosphate, hydrogenphosphate, or dihydrogenphosphate. Preference is given to salts of mono- and dicarboxylic acids, hydroxy acids, keto acids and amino acids, or basic salts. For example are acetates, propionates, tartrates, maleates, citrates, lactates, malates, succinates. Equally preferred is the use of hydroxides. Particular preference is given to the use of 2-hydroxycarbonic salts such as citrates and lactates. Examples of particularly preferred metal salts are alkali metal and alkaline earth metal aluminates and hydrates thereof, for instance sodium aluminate and hydrates thereof, aluminum acetate, aluminum propionate, aluminum citrate and aluminum lactate.

The cations and salts mentioned may be used in pure form or as a mixture of different cations or salts. The salts of the di- and/or trivalent metal cation used may comprise further secondary constituents such as still unneutralized carboxylic acid and/or alkali metal salts of the neutralized carboxylic acid. Preferred alkali metal salts are those of sodium and potassium, and those of ammonium. They are typically used in the form of an aqueous solution which is obtained by dissolving the solid salts in water, or is preferably obtained directly as such, which avoids any drying and purification steps. Advantageously, it is also possible to use the hydrates of the salts mentioned, which often dissolve more rapidly in water than the anhydrous salts.

The amount of metal salt used is generally at least 0.001% by weight, preferably at least 0.01% by weight and more preferably at least 0.1% by weight, for example at least 0.4% by weight, and generally at most 5% by weight, preferably at most 2.5% by weight and more preferably at most 1% by weight, for example at most 0.7% by weight, based in each case on the mass of the base polymer.

The salt of the trivalent metal cation can be used in the form of a solution or suspension. The solvents used for the metal salts may be water, alcohols, DMF, DMSO, and mixtures of these components. Particular preference is given to water and water/alcohol mixtures, for example water/methanol, water/1,2-propanediol and water/1,3-propanediol.

The base polymer is treated with a solution of a divalent or polyvalent cation in the same manner as that with surface postcrosslinker, including the drying step. Surface postcrosslinker and polyvalent cation can be sprayed on in a combined solution or as separate solutions. The spray application of the metal salt solution to the superabsorbent particles can be effected either before or after the surface postcrosslinking. In a particularly preferred process, the spray application of the metal salt solution is effected in the same step as the spray application of the crosslinker solution, both solutions being sprayed on separately and successively or simultaneously through two nozzles, or crosslinker and metal salt solution may be sprayed on together through one nozzle.

Especially when a trivalent or higher-valency metal cation such as aluminum is used for complexation, a basic salt of a divalent metal cation or a mixture of such salts is also optionally added. Basic salts are salts which are suitable for increasing the pH of an aqueous acidic solution, preferably 0.1 N hydrochloric acid. Basic salts are typically salts of a strong base with a weak acid.

The divalent metal cation of the optional basic salt is preferably a metal cation of group 2 of the Periodic Table of the Elements, more preferably calcium or strontium, most preferably calcium.

The basic salts of the divalent metal cations are preferably salts of weak inorganic acids, of weak organic acids and/or salts of amino acids, more preferably hydroxides, hydrogencarbonates, carbonates, acetates, propionates, citrates, gluconates, lactates, tartrates, malates, succinates, maleates and/or fumarates, most preferably hydroxides, hydrogencarbonates, carbonates, propionates and/or lactates. The basic salt is preferably water-soluble. Water-soluble salts are salts which, at 20° C., have a water solubility of at least 0.5 g of salt per liter of water, preferably at least 1 g of salt per 1 of water, preferentially at least 10 g of salt per I of water, more preferably at least 100 g of salt per l of water, most preferably at least 200 g of salt per l of water. However, it is also possible in accordance with the invention to use those salts which have this minimum solubility at the spray application temperature of the spray solution. It is advantageously also possible to use the hydrates of the salts mentioned, which often dissolve more rapidly in water than the anhydrous salts.

Suitable basic salts of divalent metal cations are, for example, calcium hydroxide, strontium hydroxide, calcium hydrogencarbonate, strontium hydrogencarbonate, calcium acetate, strontium acetate, calcium propionate, strontium propionate, calcium lactate, strontium lactate, calcium carbonate and strontium carbonate.

When the water solubility is insufficient to prepare a spray solution of the desired concentration, it is also possible to use dispersions of the solid salt in a saturated aqueous solution thereof. For example, it is possible to use calcium carbonate, strontium carbonate, calcium sulfite, strontium sulfite, calcium phosphate and strontium phosphate as aqueous dispersions.

The amount of basic salt of the divalent metal cation, based on the mass of the base polymer, is typically from 0.001 to 5% by weight, preferably from 0.01 to 2.5% by weight, preferentially from 0.1 to 1.5% by weight, more preferably from 0.1 to 1% by weight, most preferably from 0.4 to 0.7% by weight.

The basic salt of the divalent metal cation can be used in the form of a solution or suspension. Examples thereof are calcium lactate solutions or calcium hydroxide suspensions. Typically, the salts are sprayed on with an amount of water of not more than 15% by weight, preferably of not more than 8% by weight, more preferably of not more than 5% by weight, most preferably of not more than 2% by weight, based on the superabsorbent.

Preference is given to spraying an aqueous solution of the basic salt onto the superabsorbent. Conveniently, the basic salt is added simultaneously with the surface postcrosslinker, the complexing agent or as a further constituent of the solutions of these agents. For these basic salts, preference is given to addition in a mixture with the complexing agent. When the solution of the basic salt is not miscible with the solution of the complexing agent without precipitation, the solutions can be sprayed on separately in succession or simultaneously from two nozzles.

If, after the surface postcrosslinking and/or treatment with complexing agent, a drying step is carried out, it is advantageous but not absolutely necessary to cool the product after the drying step. The cooling can be effected continuously or batchwise; to this end, the product is conveniently conveyed continuously into a cooler connected downstream of the drier. To this end, it is possible to use any apparatus known for removal of heat from pulverulent solids, especially any apparatus mentioned above as a drying apparatus, provided that it is not charged with a heating medium but rather with a cooling medium, for instance with cooling water, such that no heat is introduced into the superabsorbent via the walls and, according to the construction, also via the stirrer units or other heat exchange surfaces, but rather removed therefrom. Preference is given to the use of coolers in which the product is moved, i.e. cooled mixers, for example paddle coolers or disk coolers. The superabsorbent can also be cooled in a fluidized bed by blowing in a cooled gas such as cold air. The cooling conditions are established such that a superabsorbent with the temperature desired for further processing is obtained. Typically, a mean residence time in the cooler of generally at least 1 minute, preferably at least 3 minutes and more preferably at least 5 minutes, and generally at most 6 hours, preferably at most 2 hours and more preferably at most 1 hour, is established, and the cooling performance is such that the resulting product has a temperature of generally at least 0° C., preferably at least 10° C. and more preferably at least 20° C., and generally at most 100° C., preferably at most 80° C. and more preferably at most 60° C.

The surface postcrosslinked superabsorbent is optionally ground and/or screened in a customary manner. Grinding is typically not required here, but screening-off of agglomerates or fines formed is usually appropriate to establish the desired particle size distribution of the product. Agglomerates and fines are either discarded or preferably recycled into the process in a known manner at a suitable point; agglomerates after comminution. The particle sizes desired for surface postcrosslinked superabsorbents are the same as for base polymers.

It is optionally possible to additionally apply to the surface of the superabsorbent particles, whether unpostcrosslinked or postcrosslinked, in any process step of the preparation process, if required, all known coatings, such as film-forming polymers, thermoplastic polymers, dendrimers, polycationic polymers (for example polyvinylamine, polyethyleneimine or polyallylamine), water-insoluble polyvalent metal salts, for example magnesium carbonate, magnesium oxide, magnesium hydroxide, calcium carbonate, calcium sulfate or calcium phosphate, all water-soluble mono- or polyvalent metal salts known to those skilled in the art, for example aluminum sulfate, sodium salts, potassium salts, zirconium salts or iron salts, or hydrophilic inorganic particles such as clay minerals, fumed silica, colloidal silica sols, for example Levasil®, titanium dioxide, aluminum oxide and magnesium oxide. Examples of useful alkali metal salts are sodium and potassium sulfate, and sodium and potassium lactates, citrates and sorbates. This allows additional effects, for example a reduced caking tendency of the end product or of the intermediate in the particular process step of the production process, improved processing properties or a further enhanced saline flow conductivity (SFC), to be achieved. When the additives are used and sprayed on in the form of dispersions, they are preferably used as aqueous dispersions, and preference is given to additionally applying an antidusting agent to fix the additive on the surface of the water-absorbing polymer. The antidusting agent is then either added directly to the dispersion of the inorganic pulverulent additive; optionally, it can also be added as a separate solution before, during or after the application of the inorganic pulverulent additive by spray application. Most preferred is the simultaneous spray application of postcrosslinker, antidusting agent and pulverulent inorganic additive in the postcrosslinking step. In a further preferred process variant, the antidusting agent is, however, added separately in the cooler, for example by spray application from above, below or from the side. Particularly suitable antidusting agents which can also serve to fix pulverulent inorganic additives on the surface of the water-absorbing polymer particles are polyethylene glycols with a molecular weight of from 400 to 20 000 g/mol, polyglycerol, 3- to 100-tuply ethoxylated polyols such as trimethylolpropane, glycerol, sorbitol and neopentyl glycol. Particularly suitable are 7- to 20-tuply ethoxylated glycerol or trimethylolpropane, for example Polyol TP 70® (Perstorp, Sweden). The latter have, more particularly, the advantage that they lower the surface tension of an aqueous extract of the water-absorbing polymer particles only insignificantly.

It is equally possible to adjust the inventive superabsorbent to a desired water content by adding water.

Optionally, the inventive superabsorbents are provided with further additives which stabilize against discoloration. Examples are especially known stabilizers against discoloration, especially reducing substances. Among these, preference is given to solid or dissolved salts of phosphinic acid (H₃PO₂) and to this acid itself. For example, all phosphinates of the alkali metals are suitable, including those of ammonium, and of the alkaline earth metals. Particular preference is given to aqueous solutions of phosphinic acid which comprise phosphinate ions and at least one cation selected from sodium, potassium, ammonium, calcium, strontium, aluminum, magnesium. Equally preferred are salts of phosphinic acid (H₃PO₃) and this acid itself. For example, all primary and secondary phosphonates of the alkali metals, including of ammonium, and of the alkaline earth metals are suitable. Particular preference is given to aqueous solutions of phosphinic acid which comprise primary and/or secondary phosphonate ions and at least one cation selected from sodium, potassium, calcium, strontium.

All coatings, solids, additives and assistants can each be added in separate process steps, but the most convenient method is usually to add them—if they are not added during the admixing of the base polymer with surface postcrosslinkers—to the superabsorbent in the cooler, for instance by spray application of a solution or addition in finely divided solid form or in liquid form.

The L value of the superabsorbent (CIE color number) is, in the unstored state, typically at least 75, preferably at least 80, more preferably at least 85, even more preferably at least 90, and at most 100.

The a value of the superabsorbent (CIE color number) is, in the unstored state, typically from −2.5 to +2.5, preferably from −2.0 to +2.0, more preferably from 31 1.5 to +1.5.

The b value of the superabsorbent (CIE color number) in the unstored state is typically from 0 to 12, preferably from 2 to 11.

After storage at elevated temperature under high air humidity, the inventive superabsorbent, after analysis for the L and a values, has results in the region of the samples in the unstored state, and, after 100 hours of storage, still has b values of preferably not more than 12, more preferably not more than 10, and, after 300 hours of storage, still has b values of preferably not more than 15, more preferably not more than 12. A b value above 12 is critical in feminine hygiene articles and ultra thin diapers; a b value of more than 15 is also critical even in conventional diapers, since this discoloration can be perceived by the consumer on use.

In addition, the inventive superabsorbents are substantially free of compounds which lead to unpleasant odors, especially during use.

In a preferred embodiment of the present invention, the sulfinic acid derivative is applied to the surface of the postcrosslinked polymer particles in a cooler connected downstream of the surface postcrosslinking step and/or in a separate downstream mixer, and the initiator system for preparing the polymer comprises a peroxodisulfate or peroxodiphosphate and at least one of the inventive salts of 2-hydroxy-2-sulfinatoacetic acid and of 2-hydroxy-2-sulfonatoacetic acid or free acids thereof, and optionally further coinitiators.

In a further preferred embodiment of the invention, postcrosslinkers (preferably 2-oxazolidone or N-(2-hydroxyethyl)-2-oxazolidone and/or 1,3-propanediol), organic solvent and/or cosolvent (preferably isopropanol and/or 1,2-propanediol), and optionally a surfactant (preferably sorbitan monolaurate, obtainable from many manufacturers as “Span® 20” (brand of ICI Americas Inc., Wilmington, Del., U.S.A.)) are dissolved with water and then applied by means of a spray vertical mixer (preferably a Schugi® Flexomix®) to the polymer particles by means of a two-substance nozzle or one-substance nozzle, both the mixer and a drier connected directly downstream being purged with inert gas (preferably nitrogen) such that the proportion by volume of oxygen in these units is less than 14% by volume, preferably less than 10% by volume, more preferably less than 5% by volume, most preferably less than 1% by volume. At the same time as this postcrosslinking solution, a solution of at least one sulfinic acid derivative of the formula (I) and optionally of a polyvalent metal salt or of a further additive is sprayed on via a separate feed. Alternatively, the sulfinic acid derivative can also be dissolved in the postcrosslinking solution or one of the components thereof and sprayed on together with the postcrosslinking solution.

In a further preferred embodiment of the invention, postcrosslinker (preferably 2-oxazolidone or N-(2-hydroxyethyl)-2-oxazolidone and/or 1,3-propanediol), organic solvent (preferably isopropanol), aluminum lactate, optionally a further calcium salt and optionally a surfactant (preferably sorbitan monolaurate) are dissolved with water and then applied by means of a spray vertical mixer (preferably a Schugi® Flexomix®) to the polymer particles by means of a two-substance nozzle or one-substance nozzle, both the mixer and a drier connected directly downstream being purged with inert gas (preferably nitrogen) such that the proportion by volume of oxygen in these units is less than 14% by volume, preferably less than 10% by volume, more preferably less than 5% by volume, most preferably less than 1% by volume. At the same time as this postcrosslinking solution, a solution of at least one sulfinic acid derivative of the formula (I) and optionally of a polyvalent metal salt or of a further additive is sprayed on via a separate feed. Alternatively, the sulfinic acid derivative can also be dissolved in the postcrosslinking solution or one of the components thereof and sprayed on together with the postcrosslinking solution.

In a further preferred embodiment of the invention, crosslinker (preferably ethylene glycol digylcidyl ether or 2-oxazolidone or N-(2-hydroxyethyl)-2-oxazolidone and/or 1,3-propanediol and/or 1,2-propanediol), optionally organic solvent (preferably isopropanol) and optionally a surfactant (preferably sorbitan monolaurate) are dissolved with water and then applied by means of a spray vertical mixer (preferably a Schugi® Flexomix®) to the polymer particles by means of a two-substance nozzle or one-substance nozzle, both the mixer and a drier connected directly downstream being purged with inert gas (preferably nitrogen) such that the proportion by volume of oxygen in these units is less than 14% by volume, preferably less than 10% by volume, more preferably less than 5% by volume, most preferably less than 1% by volume. During or after the cooling of the postcrosslinked polymer, in this preferred embodiment, at least one sulfinic acid derivative is applied to the polymer particles, preferably in aqueous solution.

In yet a further embodiment of the invention, postcrosslinkers (preferably selected from ethylene glycol digylcidyl ether, ethylene carbonate, β-hydroxyalkylamides, polyols, 2-oxazolidone or N-(2-hydroxyethyl)-2-oxazolidone and/or 1,3-propanediol and/or 1,2-propanediol), optionally organic solvent, and optionally a little surfactant (preferably sorbitan monolaurate) are dissolved with water and then applied by means of a spray vertical mixer (preferably a Schugi® Flexomix®) to the polymer particles by means of a two-substance nozzle or one-substance nozzle, the mixer and a drier connected directly downstream not being purged with inert gas. During or after the cooling of the postcrosslinked polymer, in this embodiment, at least one sulfinic acid derivative of the formula (I) is applied to the polymer particles. In this embodiment, at least one sulfinic acid derivative of the formula (I) is optionally additionally applied to the polymer particles before or during the postcrosslinking, in order to reduce the yellowing caused by the atmospheric oxygen.

The present invention further provides hygiene articles comprising inventive superabsorbents, preferably ultra thin diapers, comprising an absorbent layer consisting of from 50 to 100% by weight, preferably from 60 to 100% by weight, preferentially from 70 to 100% by weight, more preferably from 80 to 100% by weight, most preferably from 90 to 100% by weight, of inventive superabsorbents, excluding, of course, the shell of the absorbent layer.

The inventive superabsorbents are also very particularly advantageous for production of laminates and composite structures, as described, for example, in US 2003/0181115 and US 2004/0019342. In addition to the hotmelt adhesives described in both documents for production of such novel absorbent structures and especially to the fibers composed of hotmelt adhesives which are described in US 2003/0181115 and to which the superabsorbent particles are bonded, the inventive superabsorbents are also suitable for producing entirely analogous structures using UV-crosslinkable hotmelt adhesives, which are sold, for example, as AC Resin® (BASF SE, Germany). These UV-crosslinkable hotmelt adhesives have the advantage of being processable even at from 120 to 140° C.; they are therefore better compatible with many thermoplastic substrates. A further significant advantage is that UV-crosslinkable hotmelt adhesives are toxicologically entirely safe and also do not cause any vaporization in the hygiene articles. A very significant advantage in connection with the inventive superabsorbents is the property of the UV-crosslinkable hotmelt adhesives of not tending to yellow during processing and crosslinking. This is especially advantageous when ultrathin or partly transparent hygiene articles are to be produced. The combination of the inventive superabsorbents with UV-crosslinkable hotmelt adhesives is therefore particularly advantageous. Suitable UV-crosslinkable hotmelt adhesives are, for example, described in EP 0 377 199 A2, EP 0 445 641 A1, U.S. Pat. No. 5,026,806, EP 0 655 465 A1 and EP 0 377 191 A2.

The inventive superabsorbent can also be used in other fields of industry in which liquids, especially water or aqueous solutions, are absorbed. These fields are, for example, storage, packaging, transport (as constituents of packaging material for water- or moisture-sensitive articles, for instance for flower transport, and also as protection against mechanical effects); animal hygiene (in cat litter); food packaging (transport of fish, fresh meat; absorption of water, blood in fresh fish or meat packaging); medicine (wound plasters, water-absorbing material for burn dressings or for other weeping wounds), cosmetics (carrier material for pharmaceutical chemicals and medicaments, rheumatic plasters, ultrasonic gel, cooling gel, cosmetic thickeners, sunscreen); thickeners for oil/water or water/oil emulsions; textiles (moisture regulation in textiles, shoe insoles, for evaporative cooling, for instance in protective clothing, gloves, headbands); chemical engineering applications (as a catalyst for organic reactions, for immobilization of large functional molecules such as enzymes, as an adhesive in agglomerations, heat stores, filtration aids, hydrophilic components in polymer laminates, dispersants, liquefiers); as assistants in powder injection molding, in the building and construction industry (installation, in loam-based renders, as a vibration-inhibiting medium, assistants in tunnel excavations in water-rich ground, cable sheathing); water treatment, waste removal, water removal (deicers, reusable sand bags); cleaning; agrochemical industry (irrigation, retention of melt water and dew deposits, composting additive, protection of forests from fungal/insect infestation, retarded release of active ingredients to plants); for firefighting or for fire protection; coextrusion agents in thermoplastic polymers (for example for hydrophilization of multilayer films); production of films and thermoplastic moldings which can absorb water (e.g. films which store rain and dew for agriculture; films comprising superabsorbents for maintaining freshness of fruit and vegetables which are packaged in moist films; superabsorbent-polystyrene coextrudates, for example for packaging foods such as meat, fish, poultry, fruit and vegetables); or as a carrier substance in active ingredient formulations (pharmaceuticals, crop protection).

The inventive articles for absorption of fluid differ from known examples in that they comprise the inventive superabsorbent.

Also found has been a process for producing articles for absorption of fluid, especially hygiene articles, which comprises using at least one inventive superabsorbent in the production of the article in question. In addition, processes for producing such articles using superabsorbents are known.

### Test Methods

The superabsorbent is tested by the test methods described below.

The standard test methods referred to as “WSP” described below are described in: “Standard Test Methods for the Nonwovens Industry”, 2005 edition, published jointly by the Worldwide Strategic Partners EDANA (European Disposables and Nonwovens Association, Avenue Eugène Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). This publication is obtainable both from EDANA and from INDA.

All measurements described below should, unless stated otherwise, be carried out at an ambient temperature of 23±2° C. and a relative air humidity of 50±10%. The superabsorbent particles are mixed thoroughly before the measurement unless stated otherwise.

### Centrifuge Retention Capacity (CRC)

The centrifuge retention capacity of the superabsorbent is determined by the standard test method No. WSP 241.5-02 “Centrifuge retention capacity”.

### Absorbency Under a Load of 0.3 PSI (AUL0.3 PSI)

The absorbency under a load of 2068 Pa (0.3 psi) of the superabsorbent is determined by the standard test method No. WSP 242.2-05 “Absorption under pressure”.

### Absorbency Under a Load of 0.7 PSI (AUL0.7 PSI)

The absorbency under a load of 4826 Pa (0.7 psi) of the superabsorbent is determined analogously to the standard test method No. WSP 242.2-05 “Absorption under pressure”, except using a weight of 49 g/cm² (leads to a load of 0.7 psi) instead of a weight of 21 g/cm² (leads to a load of 0.3 psi).

### Saline Flow Conductivity (SFC)

The saline flow conductivity of a swollen gel layer formed by the superabsorbent as a result of liquid absorption is determined under a pressure of 0.3 psi (2068 Pa), as described in EP 0 640 330 A1, as the gel layer permeability of a swollen gel layer of superabsorbent particles, the apparatus described in the aforementioned patent application on page 19 and in FIG. 8 being modified to the effect that the glass frit (40) is not used, the plunger (39) consists of the same polymer material as the cylinder (37) and now comprises 21 bores of equal size distributed homogeneously over the entire contact area. The procedure and evaluation of the measurement remain unchanged from EP 0 640 330 A1. The flow is detected automatically.

The saline flow conductivity (SFC) is calculated as follows:

SFC [cm³ s/g]=(Fg(t=0)×L0)/(d×A×WP),

where Fg(t=0) is the flow of NaCl solution in g/s, which is obtained with reference to a linear regression analysis of the Fg(t) data of the flow determinations by extrapolation to t=0, L0 is the thickness of the gel layer in cm, d is the density of the NaCl solution in g/cm³, A is the area of the gel layer in cm² and WP is the hydrostatic pressure over the gel layer in dyn/cm².

### Moisture Content of the Hydrogel (Residual Moisture, Water Content)

The water content of the water-absorbing polymer particles is determined by the standard test method No. WSP 230.2-05 “Moisture content”.

### Mean Particle Size

The mean particle size of the product fraction is determined by the standard test method No. WSP 220.2-05 “Particle size distribution”.

### CIE Color Number (L a b)

The color analysis is carried out according to the CIELAB method (Hunterlab, Volume 8, 1996, Book 7, pages 1 to 4) with a “LabScan XE S/N LX17309” colorimeter (HunterLab, Reston, U.S.A.). This method describes the colors via the coordinates L, a and b of a three-dimensional system. L indicates the brightness, where L=0 means black and L=100 white. The values of a and b indicate the positions of the color on the red/green and yellow/blue color axes respectively, where +a represents red, −a represents green, +b represents yellow and −b represents blue.

The color measurement corresponds to the three-area method according to DIN 5033-6.

### Storage Test

Measurement 1: A glass dish of internal diameter 9 cm and height 1.5 cm is overfilled with water-absorbing polymer particles and then smoothed flat with a blade over the edge, and the CIE color numbers are determined.

Measurement 2a: A glass dish of internal diameter 9 cm and height 1.5 cm is filled with 15 g of superabsorbent particles and these are then smoothed flat with a knife. The dish is then placed open into a climate-controlled cabinet heated to 65° C. with constant relative air humidity of 90%. After 7 days have passed, the dish is taken out and the contents are mixed and smoothed by stirring. After cooling to room temperature and the CIE color numbers are determined.

Measurement 2b: A glass dish of internal diameter 9 cm and height 1.5 cm is filled with 12 g of superabsorbent particles such that the bottom is covered homogeneously. The dish is then placed, covered with a glass lid, into a climate-controlled cabinet heated to 70° C. at a constant relative air humidity of 75%. After 6 days have passed, the dish is taken out, the particles are tipped onto a smooth surface and the CIE color numbers, after the sample has cooled to room temperature, are determined on the underside of the sample which is now at the top.

### EXAMPLES

### Example 1 (comparative)

The Pflugschar® paddle drier of capacity 5 l with a heating jacket (manufacturer: Gebr. Lödige Maschinenbau GmbH, Elsener-Strasse 7-9, 33102 Paderborn, Germany; Type VT 5R-MK) used as the polymerization reactor was initially charged with 206.5 g of water, 271.6 g of acrylic acid (stabilized with 0.02% by weight of hydroquinone monomethyl ether), 2115.6 g of a 37.3% by weight sodium acrylate solution (100 mol % neutralized) which had been filtered beforehand through activated carbon for the purpose of removing hydroquinone monomethyl ether, and 3.5 g of triacrylate of triethoxylated glycerol, and inertized by sparging with nitrogen for 20 minutes. The shaft of the reactor was constantly rotated at 100 revolutions per minute. The content of hydroquinone monomethyl ether, based on acrylic acid plus acrylate, the latter being counted as acrylic acid, was approx. 0.0064% by weight. The reaction mixture was cooled externally (reactor jacket through which coolant flowed) such that the subsequent initiator addition was effected at approx. 20° C. Finally, an initiator mixture composed of 0.4 g of sodium persulfate (dissolved in 12 g of water) and 0.13 g of Brüggolit® FF7 (dissolved in 10 g of water) was also added to the reactor with stirring, and the cooling was switched off. The reaction set in rapidly. From attainment of an internal temperature of the reactor of 30° C., the jacket was heated with heat carrier medium at 80° C. in order to conduct the reaction to completion as adiabatically as possible. On attainment of the maximum temperature, the product was cooled again (cooling liquid at −12° C.), and the resulting gel was thus cooled to below 50° C. and then discharged.

The gel formed was distributed onto two metal wire mesh sheets and dried at 160° C. in a forced-air drying cabinet. Subsequently, it was comminuted with an ultracentrifugal mill and the product was screened off to a particle size of from 150 to 710 μm. The base polymer thus prepared had a CRC of 35.8 g/g and an AUL 0.3 psi of 17.5 g/g.

For surface postcrosslinking, the base polymer thus prepared was coated in a Pflugschar® mixer with a heating jacket (manufacturer: Gebr. Lödige Maschinenbau GmbH, Elsener-Strasse 7-9, 33102 Paderborn, Germany; Type M5), at room temperature and a shaft speed of 450 revolutions per minute, by means of two two-substance spraying nozzles, with the following solutions:

Solution 1: 0.1% by weight of ethylene glycol diglycidyl ether (Denacol® EX-810 from Nagase ChemteX Corporation, Osaka, Japan), based on base polymer 0.60% by weight of 1,2-propanediol based on base polymer 0.75% by weight of water based on base polymer

Solution 2: 3.0% by weight of aqueous aluminum sulfate solution (26.8% by weight) based on base polymer

After the spray application, the product temperature was increased to 170° C. and the reaction mixture was kept at this temperature and a shaft speed of 80 revolutions per minute for 45 minutes. The resulting product was again allowed to cool to room temperature and screened. The surface postcrosslinked polymer (the superabsorbent) was obtained as the screening fraction with particle sizes between 150 μm and 850 μm.

### Example 2 (Comparative)

The superabsorbent produced in Example 1 was mixed homogeneously in a Pflugschar® mixer with a heating jacket (manufacturer: Gebr. Lödige Maschinenbau GmbH, Elsener-Strasse 7-9, 33102 Paderborn, Germany; Type M5) with 0.10% by weight of Sipernat® D17 at a shaft speed of 450 revolutions per minute within 20 minutes, and simultaneously coated by means of a two-substance spray nozzle with an aqueous solution of 0.08% by weight of polyethylene glycol-400 and 2.0% by weight of water (based in each case on base polymer used) at room temperature.

### Example 3 (Comparative)

The superabsorbent produced in Example 1 was mixed homogeneously in a Pflugschar® mixer with a heating jacket (manufacturer: Gebr. Lödige Maschinenbau GmbH, Elsener-Strasse 7-9, 33102 Paderborn, Germany; Type M5) with 0.10% by weight of Sipernat® D17 at a shaft speed of 450 revolutions per minute within 20 minutes, and simultaneously coated by means of a two-substance spray nozzle with an aqueous solution of 0.08% by weight of polyethylene glycol-400, 0.40% by weight of sodium bisulfite and 2.0% by weight of water (based in each case on base polymer used) at room temperature.

### Example 4 (Comparative)

The procedure was as in Example 3, except that 0.80% by weight of sodium bisulfite was used.

### Example 5 (Comparative)

The procedure was as in Example 3, except that 1.20% by weight of sodium bisulfite was used.

### Example 6 (Comparative)

The procedure was as in the preparation of the base polymer in Example 1, except that 1.29 g of triacrylate of triethoxylated glycerol, 0.618 g of sodium persulfate and, instead of the Brüggolit® FF7 in 10 g of water, 0.013 g of ascorbic acid in 9.12 g of water were used. The product obtained was additionally finally screened off to a particle size of from 200 to 600 μm. It was not surface postcrosslinked.

### Example 7

A base polymer (customary, surface nonpostcrosslinked polyacrylate superabsorbent with CRC=36 g/g, AUL 0.3 psi=16 g/g and a particle size distribution (mean values) <150 μm=0.5% by weight;>150 μm=15.8% by weight;>300 μm=70.9% by weight;>600 μm=12.8% by weight and >710 μm=0.05% by weight) was conducted through a mixer (Schugi® Flexomix® Type 100 D, manufacturer: Hosokawa Micron B. V. Gildenstraat 26, 7005 BL Doetinchem, the Netherlands) for surface postcrosslinking. The base polymer was metered in gravimetrically; the throughput was 80 kg/h. The shaft was rotated at 3400 revolutions per minute; the paddles were horizontal. At the same time, two solutions or dispersions I and II were sprayed in regulated continuous mass flow through one two-substance nozzle each onto the polymer:

Solution I was a mixture of 0.2% by weight of water, 0.8% by weight of aqueous aluminum lactate solution (a 25% by weight aqueous solution; Lohtragon® AL 250 from Dr. Paul Lohmann GmbH KG, Hauptstrasse 2, 31860 Emmerthal, Germany, www.lohmann-chemikalien.de), 0.05% by weight of 2-hydroxyethyloxazolidinone, 0.05% by weight of 1,3-propanediol, 0.5% by weight of 1,2-propylene glycol, 0.008% by weight of sorbitan monolaurate and 0.87% by weight of isopropanol, based in each case on the base polymer treated therewith. Solution I was sprayed on in an amount of 1.982 kg/h through a fine liquid nozzle (J-2850-SS type+J-73328-SS gas nozzle from Spraying Systems Deutschland GmbH, Grossmoorkehre 1, 21079 Hamburg, Germany), which was arranged at the height of the solids inlet of the Schugi® Flexomix® offset by 90° from the central axis thereof. The spray gas used was nitrogen with a pressure of 2 bar in each case.

Solution II (actually a dispersion) consisted of 0.875% by weight of water, 0.4% by weight of aluminum lactate solution (a 25% by weight aqueous solution; Lohtragon® AL 250 from Dr. Paul Lohmann GmbH KG, Hauptstrasse 2, 31860 Emmerthal, Germany, www.lohmann-chemikalien.de), 0.3% by weight of C53-80 tricalcium phosphate (Chemische Fabrik Budenheim KG, Rheinstrasse 27, 55257 Budenheim, Germany) and 0.05% by weight of Brüggolit® FF7 (L. Brüggemann KG, Salzstrasse 131, 74076 Heilbronn, Germany, www.brueggemann.com), based in each case on the base polymer treated therewith. The Brüggolit® FF7 was first dissolved in water, the aluminum lactate solution was added and then the tricalcium phosphate was dispersed in this solution with a high-speed stirrer (Ultra-Turrax® T50 with S50KR shaft, IKA® Werke GmbH & Co. KG, Janke & Kunkel-Str. 10, 79219 Staufen, Germany). In the initial charge vessel, the suspension was kept homogeneous by stirring. Solution II was sprayed on in an amount of 1.30 kg/h via a coarser liquid nozzle (J-60100-SS type+J-125328-SS gas nozzle from Spraying Systems Deutschland GmbH, Grossmoorkehre 1, 21079 Hamburg, Germany), which was arranged at the height of the solids inlet of the Schugi® Flexomix® offset by 270° relative to the central axis thereof. The spraying gas used was nitrogen with a pressure of in each case 2 bar.

The moist polymer was transferred directly from the Schugi® Flexomix® mixer falling into a steam-heated paddle drier (NPD 1.6 W type, purchased from Nara Machinery Co., Ltd., European branch, Europaallee 46, 50226 Frechen, Germany). The temperature in the drier was adjusted such that a target value of the product temperature at the drier outlet of 187° C. was attained. The adjustment of the drier with a discharge direction inclination of 3°, a weir height of approx. 64 mm, which corresponds to a fill level of approx. 95%, and a rotation of the shaft of approx. 14 rpm established a mean residence time of the product in the drier of approx. 35 minutes. Connected downstream of the drier was a screw cooler of capacity 50 l (manufacturer: Lurgi Gesellschaft für Wärmetechnik mbH, Frankfurt/Main, Germany), in which the product was cooled to approx. 50° C. Subsequently, the product was passed through a screening machine equipped with 2 screening decks (150 μm/710 μm), which removed approx. 3% by weight of polymer (based on base polymer used), predominantly as coarse material. The superabsorbent of screen fraction 150-710 μm was removed as the desired product.

### Example 8

### Superabsorbent of Example 7, Aftertreated with Brüggolit® FF7

The superabsorbent from Example 7 was conducted for a second time in the same way through the same apparatus for surface postcrosslinking described in Example 7, with the same parameters having been established, except that, in the Schugi® Flexomix®, only a solution of 0.95% by weight of water and 0.05% by weight of Brüggolit® FF7, based on the weight of the superabsorbent, was sprayed on through the nozzle arranged in the 90° position, and the temperature in the paddle drier was adjusted such that a target value of the product temperature at the outlet of 100° C. was achieved.

### Example 9

### Superabsorbent, With Aluminum Lactate and Brüggolit® FF7

Example 7 was repeated, except that, in the Schugi® Flexomix®, only one solution was sprayed on in equal parts through two nozzles, and additionally using identical nozzles (J-2850-SS type+J-73328-SS gas nozzle). The composition of the solution sprayed on was: 0.76% by weight of water, 0.075% by weight of Brüggolit® FF7, 1.8% by weight of aluminum lactate solution (25% aqueous solution; Lohtragon® AL 250), 0.05% by weight of 2-hydroxyethyloxazolidinone, 0.5% by weight of 1,2-propylene glycol, 0.008% by weight of sorbitan monolaurate and 0.91% by weight of isopropanol, based in each case on the base polymer used.

### Example 10 (Comparative)

Example 7 was repeated, except that no Brüggolit® FF7 was added during the surface postcrosslinking.

### Example 11

A solution of 40.8 g of water, 0.12 g of Brüggolit® FF7 and 0.72 g of calcium lactate was sprayed at room temperature onto 1200 g of the superabsorbent prepared in Example 10 in a Pflugschar® mixer with a heating jacket (manufacturer: Gebr. Lödige

Maschinenbau GmbH, Elsener-Strasse 7-9, 33102 Paderborn, Germany; M5 type) at a shaft speed of 450 revolutions per minute by means of a two-substance spray nozzle within 2 minutes. Subsequently, the resulting polymer was dried in a forced-air drying cabinet at 100° C. for 60 minutes. By means of a screen of mesh size 850 μm, coarse fractions were removed.

The superabsorbents of Examples 1 to 11 were subjected to the storage test. Color numbers were obtained according to measurement 1a and measurement 2b. The results are compiled in the following table:

The absorption properties of the superabsorbents from Examples 1-11 were as follows:

| | CRC | AUL 0.7 psi | SFC |
|---|---|---|---|
| Example | [g/g] | [g/g] | [10⁻⁷ cm³s/g] |
| 1 (comparative) | 32.9 | 20.6 | 22 |
| 2 (comparative) | 32.6 | 20.2 | 26 |
| 3 (comparative) | 32.5 | 19.8 | 28 |
| 4 (comparative) | 32.8 | 20.0 | 24 |
| 5 (comparative) | 32.4 | 19.7 | 21 |
| 6 (comparative) | 35.6 | <10 g/g | not determined |
| 7 | 28.1 | 24.3 | 130 |
| 8 | 29.0 | 24.4 | 100 |
| 9 | 28.8 | 25.4 | 117 |
| 10 (comparative) | 28.4 | 24.2 | 109 |
| 11 | 29.2 | 24.3 | 99 |

The superabsorbents of Examples 1-11 were subjected to the storage test. The results are compiled in the table below:

| | | | | After storage, | | |
|---|---|---|---|---|---|---|
| | Before storage, | | | measurement 2a (Ex. 2-5) | | |
| | measurement 1 | | | or 2b (Ex. 1, 6-11) | | |
| Example | L | a | b | L | a | b |
| 1 (comparative) | 91.12 | −0.85 | 9.66 | 87.16 | −0.18 | 9.18 |
| 2 (comparative) | 89.4 | −1.1 | 3.9 | 79.2 | 1.5 | 6.9 |
| 3 comparative) | 89.0 | −1.6 | 6.5 | 83.8 | 0.1 | 4.1 |
| 4 (comparative) | 88.4 | −1.6 | 7.7 | 83.8 | 0.0 | 4.7 |
| 5 (comparative) | 89.9 | −1.7 | 7.3 | 84.3 | 0.1 | 4.5 |
| 6 (comparative) | 88.76 | −0.42 | 11.35 | 76.49 | 3.48 | 13.84 |
| 7 | 90.96 | −1.07 | 10.4 | 84.69 | 2.42 | 8.49 |
| 8 | 90.59 | −1.05 | 10.47 | 86.38 | 0.30 | 11.51 |
| 9 | 92.19 | −1.11 | 9.78 | 90.73 | 0.33 | 9.56 |
| 10 (comparative) | 92.75 | −0.85 | 9.78 | 81.53 | 3.38 | 11.59 |
| 11 | 91.82 | −1.03 | 9.46 | 87.86 | −0.13 | 9.58 |

The comparison of the measurements of Examples 2-5 shows significant discoloration of the samples in the course of storage, the samples treated with sodium hydrogensulfite according to the prior art being stabilized significantly to this discoloration, but the SFC value decreasing somewhat at the comparatively high hydrogensulfite content of Example 5.

A comparison of Examples 6 (without sulfinate initiator) and 1 (with sulfinate initiator) shows that, even the use of sulfinic salt as part of the redox initiator according to the prior art does not bring about any improvement in the color stability, even though no further initiator is present in the product after the polymerization. However, the inventive addition of a sulfinic acid derivative after the polymerization shows another improvement in color stability.

1. A superabsorbent comprising a compound of the formula (I) 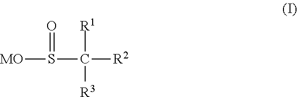
in which
M is a hydrogen atom, an ammonium ion, a monovalent metal ion, or one equivalent of a divalent metal ion of groups 1, 2, 8, 9, 10, 12, or 14 of the periodic table of the elements;
R¹ is OH or NR⁴R⁵, where R⁴ and R⁵ are each independently H or C₁-C₆-alkyl;
R² is H or an alkyl, alkenyl, cycloalkyl, or aryl group, where this group optionally has 1, 2, or 3 substituents which are each independently selected from C₁-C₆-alkyl, OH, O—C₁-C₆-alkyl, halogen, and CF₃; and
R³ is COOM, SO₃M, COR⁴, CONR⁴R⁵, or COOR⁴, where M, R⁴, and R⁵ are each as defined above or, when R² is aryl which is optionally substituted as specified above, are also H,
salts thereof or mixtures of such compounds and/or salts thereof.

2. The superabsorbent according to claim 1, wherein M is an alkali metal ion or one equivalent of an alkaline earth metal or zinc ion; R¹ is a hydroxyl or amino group; R² is H or alkyl, and R³ is COOM or COOR⁴, where, when R³ is COOM, M in this COOM radical is H, an alkali metal ion, or one equivalent of an alkaline earth metal ion, and, when R³ is COOR⁴, R⁴ is C₁-C₆-alkyl.

3. The superabsorbent according to claim 1, wherein M is an alkali metal ion or one equivalent of an alkaline earth metal or zinc ion; R¹ is a hydroxyl or amino group; R² is aryl which is optionally substituted, and R³ a hydrogen atom.

4. The superabsorbent according to claim 1, which comprises a mixture of the sodium salt of 2-hydroxy-2-sulfinatoacetic acid, the disodium salt of 2-hydroxy-2-sulfonatoacetic acid, and sodium bisulfate.

5. The superabsorbent according to claim 1, which comprises at least 0.0001% by weight and at most 3% by weight of compound of the formula (I), salts thereof or mixtures of such compounds and/or salts thereof, based in each case on the total weight of the superabsorbent.

6. The superabsorbent according to claim 1, which is surface post-crosslinked.

7. The superabsorbent according to claim 1, which further comprises a phosphinate and/or a phosphonate.

8. A process for producing a superabsorbent defined in claim 1, comprising mixing of a compound of the formula (I), salts thereof or mixtures of such compounds and/or salts thereof, and optionally a phosphinate and/or a phosphonate, with a superabsorbent.

9. An article for absorbing fluids, comprising a superabsorbent defined in claim 1.

10. A process for producing articles for absorbing fluid, which comprises using a superabsorbent defined in claim 1 in the production.
